(19) United States
(12) Patent Application Publication
(10) Pub. No.: US 2009/0264798 A1
(43) Pub. Date: Oct. 22, 2009

(54) SYSTEM AND METHOD FOR CONTROLLING ENERGY DELIVERY USING LOCAL HARMONIC MOTION

(76) Inventors: Kullervo Hynynen, Toronto (CA); Laura Curiel, Toronto (CA)

Correspondence Address: ZIOLKOWSKI PATENT SOLUTIONS GROUP, SC (ZPS), 136 S WISCONSIN ST, PORT WASHINGTON, WI, 53074, US

(21) Appl. No.: 12/104,071

(22) Filed: Apr. 16, 2008

(51) Int. Cl. A61N 7/00; A61H 1/00; A61F 7/00

(52) U.S. Cl. 601/2; 607/96

(57) ABSTRACT

A system and method for control of energy application to a target location based on a measured localized harmonic motion is disclosed. The system includes a first energy source configured to deliver a beam of energy to a subject to induce mechanical vibration of a desired region, a second energy source configured to deliver a second beam of energy into the desired region, and a receiver configured to receive echo signals from the desired region indicative of reflected energy from the second energy source. The system also includes a computer programmed to analyze at least one of amplitude, phase, and frequency of the vibration of the desired region indicated by the received echo signals, monitor the amplitude, phase, and/or frequency of the vibration in the desired region during application of the beam of energy, detect a change in the amplitude, phase, and/or frequency of the vibration in the desired region and, if the change exceeds a pre-determined size and rate, generate an alert.

### GOVERNMENT LICENSE RIGHTS

The present invention was made at least in part with Government support under Grant No. NIH CA102884, awarded by the National Institutes of Health. The Government has certain rights in the invention.

### BACKGROUND OF THE INVENTION

The present invention relates generally to a system and method of energy delivery and, more particularly, to the monitoring and control of energy delivery using localized harmonic motion measurements of a target location.

Focused ultrasound therapy involves delivering ultrasound energy to localized regions of tissue from externally (non-invasive) or internally (minimally-invasive) located transducers. The amount of ultrasound energy delivered to tissue dictates the nature of the biologic effect produced at that location. At high intensities with continuous exposure, ultrasound energy can generate enough heat to cause irreversible thermal damage through coagulation (i.e., lesion formation).

The temperature elevation induced by ultrasound in vivo depends on local properties of the tissues that determine the energy absorption and the heat transfer induced by thermal conduction and blood perfusion. These properties can vary significantly between different tissues and within a target treatment volume. Even if the same treatment parameters are applied each time, the local properties of the tissue can lead to a potential variation in clinical results. One way to eliminate this uncertainty is to monitor and control the temperature elevation and thermal dose during the treatment. Magnetic resonance imaging (MRI) can provide temperature monitoring within tissues during a treatment, making this modality an effective choice as a treatment control tool. However, the cost involved in MRI-controlled treatments is high, making the search of lower cost alternatives an important goal.

One tissue property that has shown potential for use in monitoring focused ultrasound surgery is stiffness. It has been shown that tissue stiffness is a function of temperature, and that tissue stiffness decreases initially during heating and starts to increase if heated above a certain temperature threshold, thus suggesting a tissue and temperature-dependent irreversible protein denaturation process. As the change of tissue stiffness is directly related to thermal-induced coagulation, it can be used as an indicator that adequate thermal exposure was reached. Thus, during a focused ultrasound surgery procedure, the temperature dependence of tissue stiffness provides for a reliable indicator that can be used to monitor and control the temperature elevation and thermal dose application.

In an attempt to make use of this temperature dependence of tissue stiffness, different techniques have been implemented for estimating stiffness-related parameters within tissues, such as via strain measurements, tissue displacement under a localized force, response to vibration, and ultrasound-stimulated acoustic emission (USAE) of tissues. Techniques such as acoustic radiation force impulse imaging, vibro-acoustography, ultrasound-based elastography, and magnetic resonance elastography have attempted to estimate stiffness-related parameters within tissues based on these parameters. However, each of the above techniques has been shown to have its limitations. For instance, some of the above mentioned techniques are difficult to perform in vivo in a clinical application for measurement of tissue stiffness-related parameters. Additionally, some of the above mentioned techniques are dependent on a tissue response of surrounding tissue rather than the tissue located at the focused ultrasound target location and only allow for periodic data acquisition in the monitored tissue.

It would therefore be desirable to have a system and method that provides for the accurate and continuous monitoring of focused ultrasound induced temperature elevation in vivo by using localized harmonic motion measurements of target tissue. It is further desired that such a system and method also allows for the monitoring and controlling of thermal dose application to the target tissue based on the localized harmonic motion measurements of the target tissue.

### BRIEF DESCRIPTION OF THE INVENTION

Embodiments of the present invention provide a system and method that overcome the aforementioned challenges by providing for the control of energy application to a target location based on a measured localized harmonic motion. The localized harmonic motion of the target location is monitored during a procedure, and the application of the energy is controlled based on amplitude, phase, and frequency characteristics of the localized harmonic motion so as to bring about a desired change in the target location.

In accordance with one aspect of the invention, an energy delivery system includes a first energy source configured to deliver at least one beam of energy to a desired region in a subject to induce temperature elevation and mechanical vibration of the desired region, a second energy source configured to deliver a second beam of energy into the desired region, and a receiver configured to receive echo signals from the desired region that are indicative of reflected energy from the second energy source. The energy delivery system also includes a computer programmed to analyze at least one of amplitude, phase, and frequency of the vibration of the desired region indicated by the received echo signals and monitor the at least one of amplitude, phase, and frequency of the vibration in the desired region during application of the at least one beam of energy. The computer is further programmed to detect a change in the at least one of amplitude, phase, and frequency of the vibration in the desired region and, if the change exceeds at least one of a pre-determined size and rate, generate an alert.

In accordance with another aspect of the present invention, a method of controlling energy delivery to a target location in an object includes the steps of delivering a primary energy from one or more primary sources into a target location in an object to induce temperature elevation and vibrations of the target location and transmitting a secondary energy from a secondary source into the target location, the secondary energy comprising detection bursts of energy. The method also includes the steps of receiving signals from the target location in response to the detection bursts, analyzing a component of the vibrations of the target location, monitoring the component of the vibrations during delivery of the primary energy to detect a condition change in the target location, and altering delivery of primary energy from the one or more primary sources upon detection of the condition change at the target location.

In accordance with yet another aspect of the present invention, a computer readable storage medium includes a computer program stored thereon for controlling energy delivery to a desired region in an object. The computer program comprises instructions that, when executed by a computer, cause the computer to request transmission of a first energy to the desired region from a first energy source, the first energy configured to induce temperature elevation and vibration of the desired region. The instructions further cause the computer to request transmission of a second energy to the desired region from a second energy source to generate signals corresponding to the vibration of the desired region, receive the signals corresponding to the vibration of the desired region, monitor the signals over a period in which the first energy is transmitted to the desired region, and modify transmission of the first energy from the first energy source based on the monitoring.

Various other features and advantages of the present invention will be made apparent from the following detailed description and the drawings.

### BRIEF DESCRIPTION OF THE DRAWINGS

The drawings illustrate embodiments presently contemplated for carrying out the invention.

In the drawings:

FIG. 1 is a schematic block diagram of an energy delivery system according to an embodiment of the present invention.

FIG. 2 is a schematic block diagram of an energy delivery system according to another embodiment of the present invention.

FIG. 3 is a flow chart of a process of controlling energy application to a target location using localized harmonic motion data.

FIG. 4(a) is a plot showing tissue vibration displacement before lesion formation in the tissue.

FIG. 4(b) is a plot showing tissue vibration displacement after lesion formation in the tissue.

### DETAILED DESCRIPTION OF THE PREFERRED EMBODIMENT

According to the present invention, an energy delivery system is provided that allows for the control of energy delivery to a target location based on measured localized harmonic motion. While described herein as a focused ultrasound system that provides focused ultrasound to a target tissue to induce thermal coagulation, it is recognized that the present invention is also suitable for the controlling of a delivery of various forms of energy to other animate or inanimate objects.

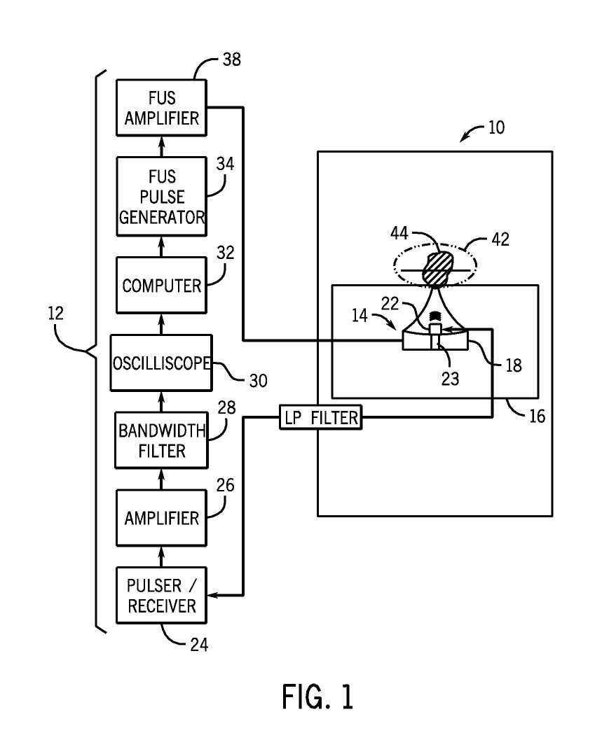

Referring to FIG. 1, a focused ultrasound (FUS) system 10 includes an ultrasound generator/receiver subsystem 12, a transducer set 14, and a tank (or other coupling technique) 16 filled, for example, with degassed water. A tank may not be needed if coupling between the transducer and a subject is achieved in another manner (for example, direct coupling with the skin). It is also envisioned that other low attenuation mediums/mechanisms can comprise the coupling element, such as ultrasound gel or a flexible water coupling.

The transducer set 14 includes a focused ultrasound (FUS) transmitting transducer 18 (i.e. a first/primary energy source) and a diagnostic transducer 22 (i.e., second energy source). The set 14 may be a phased array of transducers, with the transducer 18 being a portion of the phased array. The diagnostic transducer 22 may also be implemented with one or more transducers, including being a portion of the phased array of which the FUS transmitting transducer 18 is part. It is also envisioned that the functions of FUS transducer 18 and diagnostic transducer 22 can be combined in a single transducer. In one embodiment, FUS transducer 18 includes a central hole 23 therein in which diagnostic transducer 22 is mounted, such that the focal volume of transducer 22 is aligned with that of the FUS transducer 18 and the signal-to-noise ration (SNR) is optimized. The subsystem 12 includes a pulser/receiver 24, an amplifier 26, a bandpass filter 28, an oscilloscope 30, a personal computer 32, a FUS pulse generator 34, and a FUS amplifier 38. The system 10 is configured to image a subject 42 and more particularly a target region 44 in the subject 42.

The computer 32, the FUS pulse generator 34, and the FUS amplifier 38 of the subsystem 12 are configured to provide excitation signals for the FUS transducer 18. The computer 32 is configured to drive the FUS pulse generator 34 to produce an excitation signal that is modulated in amplitude with a desired repetition frequency (e.g., 50 to 300 Hz). The modulation could be done, for example, in the form of a 50% duty cycle burst or a sinusoidal modulation so as to selectively provide the transducer 18 with an excitation signal, although it is envisioned that other suitable duty cycles could also be implemented. The operation of FUS pulse generator 34 at a 50% duty cycle (i.e., 50% on-50% off) or at a modulated amplitude causes the FUS transducer 18 to apply a harmonic radiation force, F₀, to the target region 44 that results in tissue motion being produced (i.e., vibration). More specifically, a localized harmonic motion (LHM) is produced in the tissue at target region 44 in response to the pulsed ultrasound energy delivered by FUS transducer 18. The amplitude of the LHM in target region 44 is dependent upon various factors, such as the mechanical and acoustical properties of the target region 44 and the temperature of the target region, as will be discussed in greater detail below.

In addition to producing motion in the tissue at target region 44, the ultrasound signal produced by FUS transducer 18 is of such a frequency, amplitude, and duration that a localized temperature elevation is achieved in the tissue at target region 44. The localized temperature elevation should be of such an amount that tissue destruction is achieved via thermal-induced coagulation (i.e., lesion formation). Thus, for example, the ultrasound signal focused at target region 44 can be provided at a central frequency, f₁, of 1.5 to 1.7 MHz and at an acoustical power of 22.5 W. As set forth above, the ultrasound signal is applied at a burst repetition frequency controlled by FUS pulse transmitter 34 at, for example, 100 Hz and for a time period sufficient for inducing tissue coagulation (e.g., for a duration of 40 seconds).

In addition to driving the delivery of focused ultrasound energy from FUS transducer 18, the subsystem 12 also provides pulse excitation signals for the diagnostic transducer 22. The pulser/receiver 24 of subsystem 12 operates the transducer 22 at a pulse/receive frequency, e.g., 3 kHz, and includes appropriate switching circuitry to selectively provide the transducer 22 with excitation pulses and to receive the RF-signals from the transducer 22. The pulser/receiver 24 is configured such that it can cause the transducer 22 to send the ultrasound pulses/bursts toward the target region 44 while the FUS transducer 18 is transmitting an energy beam to the target region 44. The transducer 22 should be configured to provide a well-collimated or focused beam to the target region 44, and as such, can comprise a plurality of transducers in the set 14 if needed. For example, the transducer 22 may be driven to provide ultrasound at an odd harmonic (e.g., third, fifth, etc.) of the set 14, if the transducers 18, 22 are portions of the set 14, (i.e., a phased array).

The diagnostic transducer 22 is aimed at the target region 44 of the subject 42 such that the pulses provided by the transducer 22 are incident upon the target region 44 that is vibrating at the frequency f₁. Echoes, as a function of time from the target region 44 due to the pulses/bursts from the diagnostic transducer 22, provide information regarding the tissue motion (i.e., amplitude and frequency) of the target region 44 substantially independently of the surroundings of the target region 44.

Portions of the subsystem 12 are configured to receive and process the echoes from the target region 44. The pulser/receiver 24 is configured to detect the echoes and can acquire tissue displacement data from these echoes for target region 44 over data acquisition windows of, for example, 20 ms during the focused ultrasound exposure of target region 44. Such a window length allows for the acquisition of between 1 and 6 periods of oscillatory motion of the target region 44 within a single window, providing sufficient displacement data for analyzing of amplitude/frequency characteristics of the LHM.

Upon receiving the echoes, pulser/receiver produces RF signals in response to the echoes and passes the responses to the amplifier 26. The amplifier 26 amplifies the received signals and provides the amplified signals to the bandpass filter 28. The signals that pass through the filter 28 are digitized by a high speed digital converter, such as the oscilloscope 30, and subsequently relayed to computer 32 for storage and analysis thereby. The computer 32 is configured to process the received, amplified, filtered, and digitized reflected signals so as to perform RF signal tracking using cross-correlation techniques with a window on the order of 1-2 mm. This window represents a signal length (or duration) to be cross-correlated before and after motion has occurred and has been found to be sufficient to produce precise time delay or displacement estimates. This signal duration corresponds to approximately 2 mm travel by the ultrasound pulse in the tissue. The computer 32 estimates displacement of the target region 44 relative to the target's initial position (i.e., before application of the radiation force) during the application of the radiation force.

Based on the measured amplitude and/or frequency of the LHM vibrations in target region 44 during sonication by FUS transducer 18, computer 32 is able to determine the state of the tissue at the target location. More specifically, the amplitude and frequency of the vibrations in the tissue at target region 44 are indicative or representative of a thermal state of the tissue and of thermal-induced coagulation and lesion formation that may be occurring at the target region 44. The measured amplitude and frequency of the LHM can be compared against one of several pre-determined threshold values to determine whether or not a desired change in the tissue at target region 44 has occurred (i.e., thermal coagulation). In one embodiment, the amplitude of the LHM is monitored and if a decrease in magnitude in this amplitude is measured that is greater than a pre-determined decrease threshold, the computer 32 determines that thermal coagulation has occurred in target region 44. The threshold setting for the decrease can be set by an operator to a desired value, and can for example, be set at a level where the decrease is larger than a noise level in the measured (echo) signal. It is also envisioned, however, that the threshold level can be set to any desired amount by the operator (e.g., 15-20 micrometers) that is suggestive of lesion formation.

Alternative to, or in combination with, monitoring of the LHM amplitude, it is also recognized that the wave form of the LHM can be monitored to detect a change of state in the tissue at target region 44. That is, monitoring of the frequency of the LHM allows for detection of a phase shift in the oscillations of the LHM motion. Such a phase shift is indicative of a change in state in the tissue at target region 44 (i.e., thermal coagulation), as the vibration of the tissue at target region 44 (in response to the FUS applied by transducer 18) becomes delayed as thermal coagulation of the tissue is induced, as compared to vibration of the tissue in its pre-sonication state. A threshold value for a phase shift can thus be set by an operator, which when exceeded, is understood as being indicative of thermal coagulation at the tissue of target region 44.

In response to the frequency/amplitude of the LHM exceeding a threshold value, the computer 32 is programmed to generate an alert. The alert can comprise an audible or visual alert that allows an operator to take a desired action, such as adjusting the operation of FUS transducer 18. Alternatively, the alert can comprise a control signal that automatically causes computer 32 to control/alter operation of the FUS transducer 18. That is, if a pre-determined threshold value for the amplitude and/or phase of the LHM is crossed during a sonication period, computer 32 acts to alter the operation of FUS transducer 18. For example, computer 32 can act to reduce or terminate transmission of a focused ultrasound beam from FUS transducer 18 immediately upon identification of the LHM amplitude/phase shift being outside of a pre-determined threshold. Alternatively, a pre-determined time period (e.g., 5 sec) can be allowed to pass after identification of the LHM amplitude/phase being outside of a pre-determined threshold before the FUS transducer is powered down, so as to allow for formation of a larger lesion at target region 44.

Computer 32 is further programmed to change an operational state of FUS transducer 18 in the event that no threshold value is crossed during a sonication period. That is, when the amplitude and/or frequency (i.e., a phase shift in the frequency) of the LHM does not cross a threshold limit within a pre-determined time period of target region 44 sonication, computer 32 acts to alter the energy transmission of FUS transducer 18. For example, the acoustical power and/or frequency of the energy beam emitted by FUS transducer 18 can be increased to induce thermal coagulation in the target region 44. This pre-determined time limit (e.g., 40 sec) can be selected by an operator, and will vary based on the original power/frequency of the applied ultrasound beam.

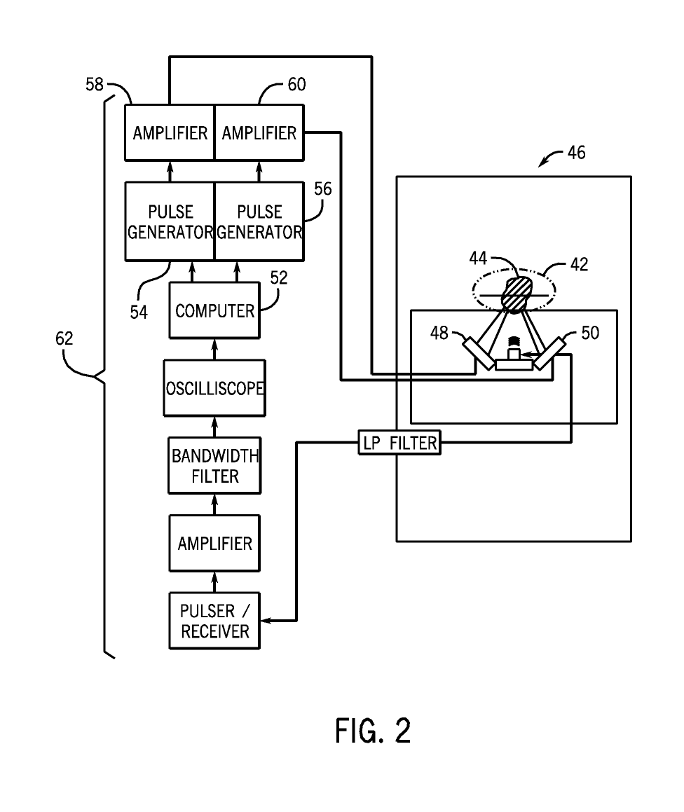

Referring now to FIG. 2, in another embodiment of the invention, a focused ultrasound (FUS) system 46 includes two FUS transducers 48, 50 that are paired to together to induce vibration (i.e., LHM) in the tissue of target region 44. A computer 52, FUS pulse generators 54, 56, and FUS amplifiers 58, 60 of subsystem 62 are configured to provide excitation signals for the FUS transducers 48, 50. The computer 52 is configured to drive the FUS pulse generators 54, 56 to produce signals of frequencies f₁, f₂, respectively, that are in the RF range and that differ from each other by preferably 10 to 5000 Hz. The FUS amplifiers 58, 60 are configured to amplify the signals from the FUS pulse generators 54, 56 to produce the excitation signals for the FUS transducers 48, 50.

The excitation signals provided by the subsystem 12 cause the FUS transducers 48, 50 to produce RF ultrasound signals focused at the target region 44 of the subject 42. Ultrasound beams from the FUS transducers 48, 50 are directed to focus and preferably intersect at the target region 44 in the subject 42. Ultrasound produced by the transducers 48, 50 is at the differing frequencies, f₁, f₂. Thus, a frequency difference, f_{d}, is described as f_{d}=f₁−f₂. The frequency difference produces a radiation force F₀ in the target region 44 at the frequency f_{d}, that results in tissue motion being produced. The tissue motion produced due to the vibration at the difference frequency f_{d} is dependent upon various factors such as the mechanical and acoustical properties of the target region 44. Additionally, the ultrasound beams are also designed to induce a localized temperature elevation in the tissue at target region 44. That is, the amplitude and frequencies (f₁, f₂) of the ultrasound beams are such that tissue destruction can be achieved via thermal-induced coagulation (i.e., lesion formation) in target region 44.

While the FUS transducers (for inducing vibration and heating the tissue) and diagnostic transducers (for generating echoes indicative of the tissue motion) of the FUS systems 10, 46 shown in FIGS. 1 and 2 are shown as separate transducers, it is also envisioned that the functions of these transducers can be performed by a single ultrasound transducer. That is, it is envisioned that a single ultrasound transducer can be controlled to transmit modulated ultrasound energy that induces motion in the target tissue and heats the tissue (i.e., “therapy energy”) and also transmit ultrasound pulses/bursts (i.e., “detection bursts”) toward the target region to generate echoes indicative of the induced motion and allow for tracking of the motion. The detection bursts can be at the same frequency or at an odd harmonic of the fundamental therapy energy. Additionally, the detection bursts can be transmitted concurrently with or interleaved with the therapy/motion inducing sonications, or they can be superimposed with the therapy/motion inducing sonications when higher harmonics are used. It is also envisioned that separate transducers could be used for inducing vibration, heating the tissue, and generating echoes indicative of the tissue motion. That is, energy (e.g., ultrasound energy) could be applied from each of three individual transducers to bring about tissue vibration, heating of the tissue, and generation of echoes indicative of the tissue motion, respectively.

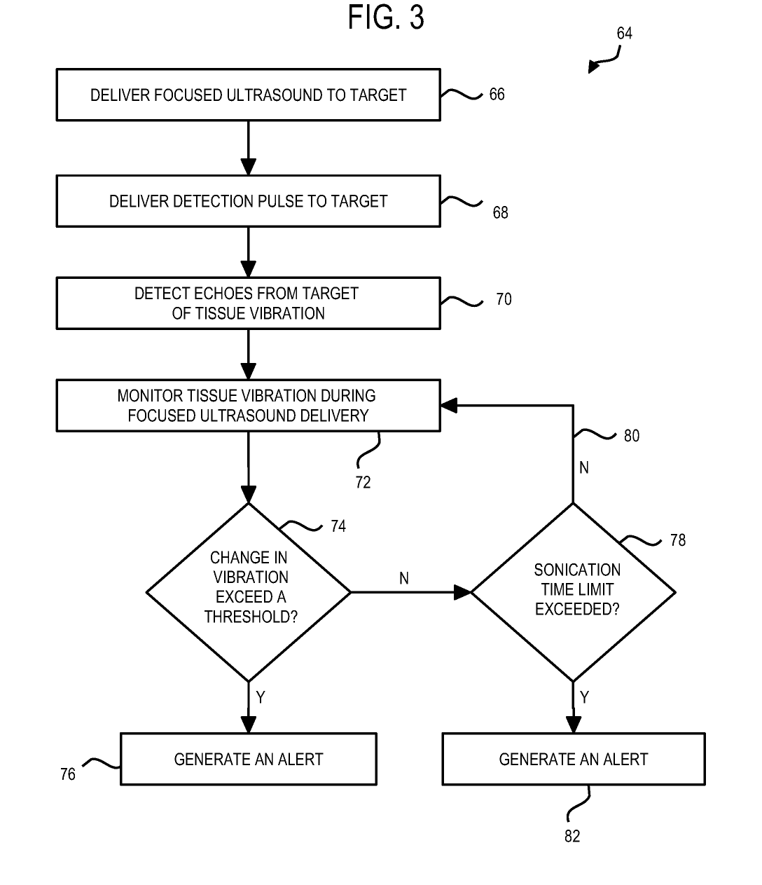

Referring now to FIG. 3 (and with further reference to FIG. 1), a technique 64 for the monitoring and controlling of thermal dose application to the target tissue (using the system 10) is set forth. The technique 64, however, is merely exemplary only and may be altered, as one skilled in the art will readily recognize. While the technique 64 is described with respect to the FUS system 10 of FIG. 1, a similar technique could also be described with respect to the FUS system 46 of FIG. 2.

As shown, an amplitude modulated ultrasound beam is directed or delivered to the target region 44 at block 66. The computer 32 controls the FUS pulse generator 34 to produce excitation pulses at a pre-determined pulse repetition frequency (e.g., 50-1000 Hz). This signal is amplified by the FUS amplifier 38 and supplied to the FUS transducer 18. The FUS transducer 18 converts the received electronic signals into ultrasound and transmits ultrasound energy bursts toward, and focused at, the target region 44 of the subject 42. The ultrasound beam has a central frequency, f₁, of e.g., 1.5-1.75 MHz, such that the tissue at target region 44 can be elevated in temperature a desired amount. Additionally, the energy bursts induce a localized harmonic motion (LHM) in the target region at the modulation frequency of the single beam.

A pulse is then produced and transmitted to the target region 44 at block 68. The pulser/receiver 24 sends an electronic signal to the diagnostic transducer 22. This pulse can, and preferably is, provided while the beam of frequency f₁ is inducing motion of the target region 44. The transducer 22 converts this into an ultrasound pulse and sends the pulse to the target region 44. The pulse reflects off the target region 44 to produce an echo received at the same transducer.

Multiple echoes are detected, processed, and displayed at block 70. The echoes are received by the diagnostic transducer 22, converted to electrical signals and sent to the pulser/receiver 24. These electrical echo signals are passed to the amplifier and the bandpass filter where they are amplified and filtered. The amplified, filtered echo signals are digitized by the oscilloscope 30 and are analyzed by the computer 32 to determine one or more mechanical responses or properties of the tissue at target region 44. The computer 32 determines vibrational characteristics for the target region over a period of time, including amplitude and frequency of the vibrations.

The computer 32 monitors the magnitude and frequency of the target region's vibration over a period of time of focused ultrasound sonication of target region 44, as shown at block 72. The measured amplitude and frequency of the LHM is compared against one of several pre-determined threshold values at block 74 to determine whether or not a desired change in the tissue at target region 44 has occurred (i.e., thermal coagulation). In one embodiment, the amplitude of the LHM is measured, and if the value of a decrease in this amplitude is measured that is greater than a pre-determined decrease threshold, the computer determines that thermal coagulation has occurred in target region 44. An exemplary decrease threshold may be in the range of 15 to 25 micrometers.

Alternative to, or in combination with, monitoring of the LHM amplitude, it is also recognized that the frequency of the LHM can be monitored to detect a phase shift in the LHM of the tissue at target region 44. That is, the frequency of the vibration is monitored to detect a phase shift in the oscillations of the LHM motion, which is indicative of a change in state in the tissue at target region 44 (i.e., thermal coagulation). A threshold value for a phase shift can thus be set by an operator, which when exceeded, is understood as being indicative of thermal coagulation at the tissue of target region 44. An exemplary threshold for the phase shift, θ, may be in the range of ¼ of an oscillation cycle, with the time length of the phase shift being dependent on the frequency of the applied ultrasound energy.

If the frequency and/or amplitude of the LHM is determined to exceed a threshold value, the computer 32 is programmed to generate an alert, as shown at block 76. The alert can comprise an audible or visual alert that allows an operator to take a desired action, such as adjusting the operation of FUS transducer. Alternatively, the alert can automatically cause computer 32 to control/alter operation of the FUS transducer 18. That is, if threshold value is for the amplitude and/or phase of the LHM is crossed during a sonication period, computer 32 acts to alter the operation of FUS transducer 18. For example, the computer can act to terminate transmission of the FUS beam from FUS transducer 18 immediately upon identification of the LHM amplitude/phase being outside of a pre-determined threshold, or alternatively, can employ a delayed power reduction or termination.

In the event that no threshold value is crossed during a sonication period, computer 32 is further programmed to generate an alert and change an operational state of FUS transducer 18 if a pre-determined period of time has passed. As shown at block 78, a determination is made by computer 32 as to whether a time limit for inducing a desired change of state in target region 44 has passed. If the time limit has not been reached, the computer goes back to monitoring the amplitude/frequency of vibrations in target region 44, indicated at block 80. If the pre-determined time limit has been exceeded, computer 32 generates an alert, as indicated at block 82. In response to the alert, computer 32 acts to alter the FUS transmission of FUS transducer 18. For example, the intensity/power and/or frequency of the FUS beam emitted by FUS transducer 18 can be increased to induce thermal coagulation in the target region 44. In a separate embodiment, it is also envisioned that an operator can take a desired action to adjust operation of the FUS transducer 18 rather than an automatic adjustment being made by computer 32.

Thus, a technique 64 for the control of focused ultrasound application to a target location based on a measured localized harmonic motion is set forth above. The technique provides for precise control of ultrasound application to the tissue at target region 44 such that it can be accurately determined when thermal-induced coagulation is occurring in vivo and bringing about lesion formation in the target tissue.

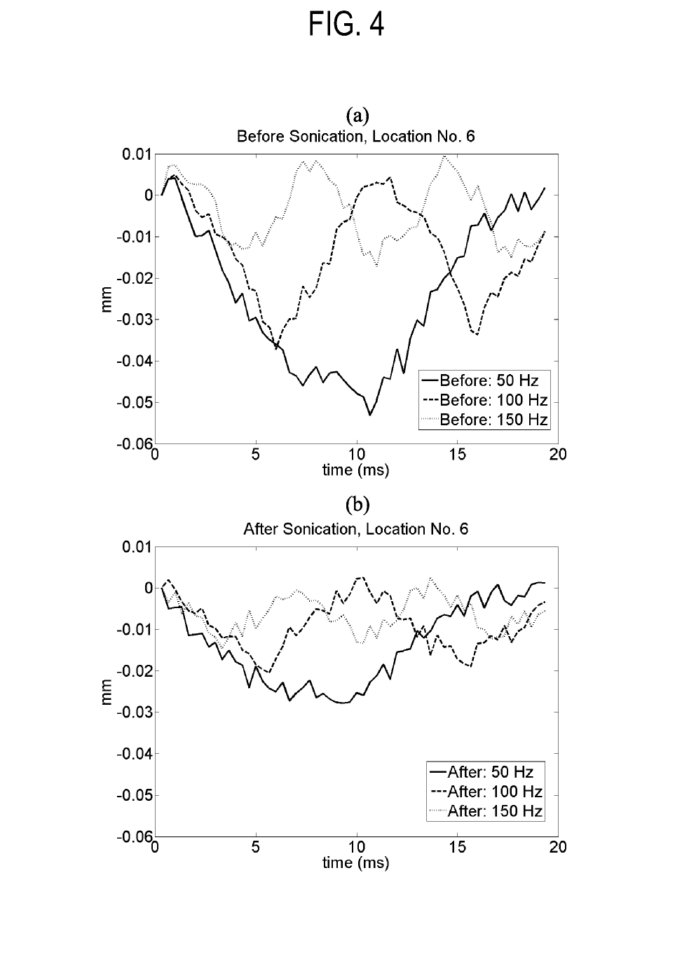

Referring now to FIGS. 4A and 4B, an example of tissue displacement (i.e., vibration amplitude) at a target location as a function of time is shown pre- and post-sonication. In FIG. 4A, tissue displacement before ultrasound sonication is illustrated for ultrasound bursts emitted at a pulse repetition frequency of 50, 100, and 150 Hz. As shown therein, the vibrational amplitude of the tissue has a maximum of approximately 0.055 mm, 0.04 mm, and 0.02 mm for each of the respectively burst frequencies. FIG. 4B illustrates tissue displacement after an ultrasound sonication procedure, such as those described in detail above. Specifically, tissue displacement for ultrasound bursts emitted at a pulse repetition frequency of 50, 100, and 150 Hz is shown. As shown in FIG. 4B, the vibrational amplitude of the tissue has a maximum of approximately 0.03 mm, 0.02 mm, and 0.01 mm for each of the burst frequencies, respectively. Thus, a comparison of the tissue displacement at a target location pre- and post-sonication shows that an amplitude of the displacement/vibration decreases after lesion formation (i.e., post-sonication) for all the frequencies.

While the above embodiments of the invention have been described with respect to ultrasound transducers and the application of ultrasound energy, it is also envisioned that various other types of energy could be applied. That is, various other types of energy could be applied to induce temperature elevation and mechanical vibration of a desired region in an object. For example, radio frequency (RF), lasers, or microwaves, can be used to deliver energy to heat the desired region and induce vibrations therein. Thus, the present invention is not limited to the application of ultrasound energy, but also encompasses additional forms of energy suitable for inducing temperature elevation and vibrations in a subject.

A technical contribution for the disclosed method and apparatus is that is provides for computer implemented control of focused ultrasound application to a target location based on a measured localized harmonic motion.

Therefore, according to one embodiment of the present invention, an energy delivery system includes a first energy source configured to deliver at least one beam of energy to a desired region in a subject to induce temperature elevation and mechanical vibration of the desired region, a second energy source configured to deliver a second beam of energy into the desired region, and a receiver configured to receive echo signals from the desired region that are indicative of reflected energy from the second energy source. The energy delivery system also includes a computer programmed to analyze at least one of amplitude, phase, and frequency of the vibration of the desired region indicated by the received echo signals and monitor the at least one of amplitude, phase, and frequency of the vibration in the desired region during application of the at least one beam of energy. The computer is further programmed to detect a change in the at least one of amplitude, phase, and frequency of the vibration in the desired region and, if the change exceeds at least one of a pre-determined size and rate, generate an alert.

According to another embodiment of the present invention, a method of controlling energy delivery to a target location in an object includes the steps of delivering a primary energy from one or more primary sources into a target location in an object to induce temperature elevation and vibrations of the target location and transmitting a secondary energy from a secondary source into the target location, the secondary energy comprising detection bursts of energy. The method also includes the steps of receiving signals from the target location in response to the detection bursts, analyzing a component of the vibrations of the target location, monitoring the component of the vibrations during delivery of the primary energy to detect a condition change in the target location, and altering delivery of primary energy from the one or more primary sources upon detection of the condition change at the target location.

According to yet another embodiment of the present invention, a computer readable storage medium includes a computer program stored thereon for controlling energy delivery to a desired region in an object. The computer program comprises instructions that, when executed by a computer, cause the computer to request transmission of a first energy to the desired region from a first energy source, the first energy configured to induce temperature elevation and vibration of the desired region. The instructions further cause the computer to request transmission of a second energy to the desired region from a second energy source to generate signals corresponding to the vibration of the desired region, receive the signals corresponding to the vibration of the desired region, monitor the signals over a period in which the first energy is transmitted to the desired region, and modify transmission of the first energy from the first energy source based on the monitoring.

The present invention has been described in terms of the preferred embodiment, and it is recognized that equivalents, alternatives, and modifications, aside from those expressly stated, are possible and within the scope of the appending claims.

What is claimed is:

1. An energy delivery system comprising:
a first energy source configured to deliver at least one beam of energy to a desired region in a subject to induce temperature elevation and mechanical vibration of the desired region;
a second energy source configured to deliver a second beam of energy into the desired region;
a receiver configured to receive echo signals from the desired region that are indicative of reflected energy from the second energy source; and
a computer programmed to:
analyze at least one of amplitude, phase, and frequency of the vibration of the desired region indicated by the received echo signals;
monitor the at least one of amplitude, phase, and frequency of the vibration in the desired region during application of the at least one beam of energy;
detect a change in the at least one of amplitude, phase, and frequency of the vibration in the desired region; and
if the change exceeds at least one of a pre-determined size and rate, generate an alert.

2. The energy delivery system of claim 1 wherein the first energy source comprises a single ultrasound transducer, the single ultrasound transducer configured to deliver harmonic bursts of ultrasound to the desired region.

3. The energy delivery system of claim 2 wherein the single ultrasound transducer operates approximately at a 50% on-50% off duty cycle.

4. The energy delivery system of claim 2 wherein the single ultrasound transducer operates at modulated amplitude.

5. The energy delivery system of claim 1 wherein the first energy source comprises a first ultrasound transducer and a second ultrasound transducer, the first ultrasound transducer configured to deliver ultrasound energy at a first frequency and the second ultrasound transducer configured to deliver ultrasound energy at a second frequency different from the first frequency.

6. The energy delivery system of claim 1 wherein the alert comprises one of an audible alert, a visual alert, and a control signal, the control signal causing the computer to alter delivery of the at least one beam of energy from the first energy source.

7. The energy delivery system of claim 6 wherein the computer is further programmed to terminate delivery of the at least one beam of energy from the first energy source upon generation of the alert.

8. The energy delivery system of claim 1 wherein the detected change comprises at least one of a decrease in the amplitude of the vibration and a phase shift in the frequency of the vibration.

9. The energy delivery system of claim 8 wherein the decrease in amplitude comprises at least one of a decrease in the amplitude of the vibration larger than a detected noise level and a decrease in the amplitude of the vibration larger than a pre-determined decrease threshold.

10. The energy delivery system of claim 1 wherein the computer is further programmed to increase an amount of energy delivered by the first energy source if the detected change in the at least one of amplitude and phase of the vibration does not exceed at least one of the pre-determined size and rate during a pre-determined time frame.

11. The energy delivery system of claim 1 wherein the first and second energy sources comprise first and second ultrasound transducers forming a portion of a phased array of transducers.

12. The energy delivery system of claim 11 wherein the second ultrasound transducer delivers an ultrasound beam with a frequency at an odd harmonic frequency produced by other transducers in the phased array.

13. The energy delivery system of claim 1 wherein the second energy source comprises a diagnostic ultrasound transducer.

14. The energy delivery system of claim 1 wherein the target region comprises biological tissue and wherein the at least one beam of energy is configured to induce thermal coagulation in the biological tissue.

15. The energy delivery system of claim 1 wherein the first and second energy sources comprise a single ultrasound transducer, the single ultrasound transducer configured to:
deliver amplitude modulated energy to the desired region in the subject to induce temperature elevation and mechanical vibration in the desired region; and
deliver detection bursts of energy to generate echoes indicative of the mechanical vibration, the detection bursts being at a same frequency or an odd harmonic of the amplitude modulated energy.

16. The energy delivery system of claim 15 wherein the detection bursts are interleaved with the amplitude modulated energy or superimposed with the amplitude modulated energy.

17. A method of controlling energy delivery to a target location in an object, the method comprising:
delivering a primary energy from one or more primary sources into a target location in an object to induce temperature elevation and vibrations of the target location;
transmitting a secondary energy from a secondary source into the target location, the secondary energy comprising detection bursts of energy;
receiving signals from the target location in response to the detection bursts;
analyzing a component of the vibrations of the target location;
monitoring the component of the vibrations during delivery of the primary energy to detect a condition change in the target location; and
altering delivery of primary energy from the one or more primary sources upon detection of the condition change at the target location.

18. The method of claim 17 wherein delivering energy from one or more primary sources comprises delivering ultrasound energy from a single primary source.

19. The method of claim 18 wherein delivering the ultrasound energy from a single primary source comprises delivering a plurality of energy bursts at a 50% duty cycle.

20. The method of claim 18 wherein delivering the ultrasound energy from a single primary source comprises delivering amplitude modulated energy.

21. The method of claim 17 wherein delivering energy from one or more primary sources comprises:
delivering energy from a first primary source at a first frequency; and
delivering energy from a second primary source at a second frequency different from the first frequency.

22. The method of claim 17 wherein monitoring the component of the vibrations to detect a condition change in the target location comprises:
monitoring at least one of amplitude, phase, and frequency of the vibrations of the target location;
detecting a change in the at least one of amplitude, phase, and frequency of the vibrations of the target location; and
determining if the change exceeds a pre-determined threshold.

23. The method of claim 22 wherein determining if the change exceeds a pre-determined threshold comprises at least one of:
determining if a decrease in the amplitude of the vibration is larger than a detected noise level; and
determining if a decrease in the amplitude of the vibration is larger than a pre-determined amplitude decrease threshold.

24. The method of claim 22 wherein determining if the change exceeds a pre-determined threshold comprises determining if a phase shift exceeds a pre-determined phase shift threshold.

25. The method of claim 17 wherein delivering a primary energy comprises delivering focused ultrasound energy from one or more focused ultrasound transducers and wherein transmitting a secondary energy comprises delivering ultrasound detection pulses from a diagnostic transducer.

26. A computer readable storage medium having stored thereon a computer program for controlling energy delivery to a desired region in an object, the computer program comprising instructions that, when executed by a computer, cause the computer to:
request transmission of a first energy to the desired region from a first energy source, the first energy configured to induce vibration of the desired region;
request transmission of a second energy to the desired region from a second energy source to generate signals corresponding to the vibration of the desired region;
receive the signals corresponding to the vibration of the desired region;
monitor the signals over a period in which the first energy is transmitted to the desired region; and
modify transmission of the first energy from the first energy source based on the monitoring.

27. The computer readable storage medium of claim 26 having further instructions to cause the computer to reduce transmission of the first energy to the desired region if at least one of an amplitude and frequency of the vibration signal crosses a pre-determined threshold.

28. The computer readable storage medium of claim 27 having further instructions to cause the computer to reduce transmission of the first energy to the desired region if the amplitude of the vibration signal is below a pre-determined amplitude threshold.

29. The computer readable storage medium of claim 27 having further instructions to cause the computer to reduce transmission of the first energy to the desired region if a phase shift in the monitored frequency of the vibration exceeds a pre-determined phase shift threshold.

30. The computer readable storage medium of claim 26 wherein the first energy comprises one of ultrasound energy, radio frequency (RF) energy, laser energy, and microwave energy.
